(19) United States
(12) Patent Application Publication
(10) Pub. No.: US 2015/0126618 A1
(43) Pub. Date: May 7, 2015

(54) COMPOSITION REPLACING WATER WITH HYALURONIC ACID AS THE PRIMARY INGREDIENT

(71) Applicant: David E. Pollock, Miami, FL (US)

(72) Inventor: David E. Pollock, Miami, FL (US)

(21) Appl. No.: 14/535,142

(22) Filed: Nov. 6, 2014

Related U.S. Application Data
(60) Provisional application No. 61/901,232, filed on Nov. 7, 2013.

(51) Int. Cl. A61K 47/26

(52) U.S. Cl. CPC A61K 47/26
USPC 514/777

(57) ABSTRACT

A biologic formulation of 20% to 95% by weight of hyaluronic acid is provided such that the hyaluronic acid is the most common ingredient in the formulation. The formulation preferably also includes 0.1% to 1.5% by weight of a preservative, and a pH adjuster configured to provide a pH of less than 5.0 in the formulation. The hyaluronic acid is a combination of two or more different molecular weights of hyaluronic acid. The different molecular weights of hyaluronic acid are preferably cross-linked with glucosamine and ceramides.

### RELATED APPLICATION

This application claims the benefit of U.S. Provisional Application No. 61/901,232, filed on Nov. 7, 2013.

### FIELD OF THE INVENTION

The present invention relates to a biologic composition for either topical or internal use comprising Hyaluronic Acid as the primary ingredient, in place of water, promoting an enhanced formulation for hydrating dry skin, enhancing the delivery of therapeutic or active ingredients, including cosmetic, dermatological and over-the-counter monographed ingredients.

### BACKGROUND OF THE INVENTION

There are a number of biologic formulations on the market designed to provide a variety of cosmetic and therapeutic benefits with the primary ingredient being water, typically over 60 and even 70 percent. Many people ignore the water listed as the primary ingredient, thinking that water is good for the skin. However, water actually has the opposite effect by stripping the skin of natural oils, leaving the skin drier and simply dilutes the level of the functional ingredients and the overall benefits of the product.

Furthermore, the majority of these conventional formulations contain soap based emulsifiers, disrupting skin pH and skin homeostasis, as well as emulsifying and stripping a portion of the skin's lipid barrier, adversely affecting and damaging the skin, including exasperating the problem of dry skin, irritated skin, inflammation, psoriasis, eczema, acne, even accelerating the signs of aging.

Human skin is comprised of the epidermis, dermis and subcutis layers. The epidermis is comprised of several layers, starting with the stratum corneum (SC) or horny cell layer, keratinocytes layer and the basal layer.

Although the skin is very thin, the SC layer acts as a protective barrier function over the body's surface regulating body temperature, minimizing water in the body and minimizing trans-epidermal water loss (TEWL), and providing resistance and protection to undesired penetration of environmental insults, chemical irritants and other foreign substances.

The SC is comprised of two phases—the flattened dead cells, corneocytes, which are embedded in a complex lipid matrix comprised of approximately 50% ceramides, 25% cholesterol, 10% free fatty acids, plus a few other minor components (Rong-Huei, C., et al, 2008).

It is the lipid domain located between the corneocytes, within the SC, which is responsible for the barrier function of the skin. Some compare the SC layer to a brick and mortar type system, with the lipid domain between the corneocytes would act as the mortar in a brick wall.

Additionally, the epidermal surface is acidic and has been the subject of many studies on epidermal permeation and formation (Loden and Howard, eds, 2000) as to the chemistry and function of dry skin and factors involved in developing skin moisturizers.

Feingold and Elias (2000) state that the epidermal surface has been known for many years to be acidic, but the role of this acidic pH of the SC in barrier homeostasis was unknown, until barrier recovery of an acidic vs neutral pH was examined. Barrier recovery proceeded normally when exposed to solutions buffered to an acidic pH, in contrast, barrier recovery was delayed when exposed to solutions with neutral or alkaline pH (Loden and Maibach, 2000).

Skin homeostasis is maintained at an acidic pH of 4.5 to 5.5 for Caucasian skin. Studies have shown that there are racial differences in pH of the surface layers of the SC, but not at the deeper SC layers (Berasdesca, et al, 1998). Traditional topical formulations have a pH of 6.0-7.5, as such the more neutral or alkaline pH actually inhibits skin repair (Mauro, 1998).

Factors, such as pH, have to be taken into account, because the proteases which lead to orderly desquamation of horny cells within the SC are activated only at an acid pH of 4 to 5 (Kligman, A., 2000).

For these reasons, a method of penetrating the protective barrier and delivering therapeutic or active ingredients safely and effectively, and without disrupting SC pH, is an on-going challenge in topical delivery for the cosmetic and medical industry. Similar concerns can be stated for cellular barriers, membranes, and tissues within the human body.

Additionally, traditional topical formulations include petrolatum based chemicals, parabens, glycols, PEGs, synthetic fragrance, synthetic dyes and other known carcinogens. As such, the overall benefits of traditional topical compositions with therapeutic or active ingredients are minimized by the formulation properties that carry them, and even worse, the chemicals used in the topical composition can cause more damage than good.

The present invention fulfills these needs and provides other related advantages.

### SUMMARY OF THE INVENTION

The present invention is directed to a biologic formulation of hyaluronic acid used for either topical or internal applications by a human, wherein the hyaluronic acid is the most common ingredient in the formulation. In addition, water is preferably excluded from the formulation other than incidental water included in a solution of another ingredient, i.e., a solution of hyaluronic acid.

The biologic formulation comprises about 20% to 95% by weight of hyaluronic acid, which is preferably the most common ingredient in the formulation. The formulation may also include 0.1% to 1.5% by weight of a preservative, and a pH adjuster configured to provide a pH of less than 5.0 in the formulation. The preservative preferably comprises a combination of sodium benzoate and potassium sorbate, or benzyl alcohol and dehydroacetic acid. The pH adjuster preferably comprises ascorbic acid or lactic acid.

For a topical application, the preservatives are preferably provided in quantities of 0.5% to 1.5% by weight. For an internal application, the preservatives are preferably provided in quantities of about 0.1% by weight. For topical application, the pH adjuster is provided in quantities to achieve a pH of between 4.5 and 5.0. For an internal application, the pH adjuster is preferably provided in quantities to achieve a pH of less than 4.0.

The hyaluronic acid preferably comprises a mixture of two or more forms of hyaluronic acid having different molecular weights. In a particularly preferred embodiment, the hyaluronic acid comprises a combination of a first molecular weight hyaluronic acid, a second molecular weight hyaluronic acid, and a third molecular weight hyaluronic acid. The first molecular weight hyaluronic acid preferably comprises hyaluronic acid having a molecular weight less than 1,500,000 Daltons. The second molecular weight hyaluronic acid preferably comprises hyaluronic acid having a molecular weight less than 100,000 Daltons. The third molecular weight hyaluronic acid preferably comprises hyaluronic acid having a molecular weight less than 10,000 Daltons.

In particularly preferred embodiments, the first molecular weight hyaluronic acid comprises hyaluronic acid having a molecular weight between 750,000 Daltons and 1,250,000 Daltons, the second molecular weight hyaluronic acid comprises hyaluronic acid having a molecular weight between 50,000 Daltons and 75,000 Daltons, and the third molecular weight hyaluronic acid comprises hyaluronic acid having a molecular weight between 3,000 Daltons and 8,000 Daltons. The different molecular weight hyaluronic acids are preferably linked by glucosamine and ceramides.

In certain formulations, the hyaluronic acid comprises 30% to 90% of the first molecular weight hyaluronic acid, 5% to 20% of the second molecular weight hyaluronic acid, and 1% to 5% of the third molecular weight hyaluronic acid. In other formulations, the hyaluronic acid comprises 10% to 90% of the first molecular weight hyaluronic acid, 10% to 70% of the second molecular weight hyaluronic acid, and 1% to 35% of the third molecular weight hyaluronic acid.

The topical formulation may also include a rheology modifier selected from the group consisting of carbomer, cellulose, and xantham gum. The rheology modifier comprises 0.01% to 1% by weight of the formulation. The topical formulation may also include 1% to 5% by weight of a non-soap based emulsifier, and 2% to 8% by weight of emollients. The non-soap based emulsifier preferably comprises lecithin. The emollients preferably comprise a combination of caprylic/capric triglyceride, squalane, natural oils, and butters.

The topical formulation may also include 0.1% to 25% by weight of a physical exfoliator, such as crushed rice, almonds, walnuts, seeds, wax beads, or jojoba beads. It may also include 0.5% to 2% by weight of allantoin and 0.5% to 5% by weight of a monographed topical analgesic. The topical analgesic preferably comprises a strong ammonia solution.

An internal formulation may comprise between 60% and 95% by weight of hyaluronic acid, more preferably 80% to 90%. The internal formulation may also comprise botanical extracts, i.e., aloe vera and/or green tea in amounts ranging from 0.01% to 5% by weight. Glycerin may be included between 2% and 10% by weight, most preferably about 5%. Phytoceramides may also be included between 0.01% and 5%, most preferably less than 2%. Sweeteners such as Stevia may also be added in amounts less than 1%, preferably between 0.25% and 0.5% by weight. As discussed above, preservatives at about 0.1% by weight and pH adjusters to bring formulation to a pH of less than 4.0 may also be added.

Other features and advantages of the present invention will become apparent from the following more detailed description, taken in conjunction with the accompanying drawings, which illustrate, by way of example, the principles of the invention.

### DETAILED DESCRIPTION OF THE PREFERRED EMBODIMENTS

The present invention provides improvements over existing formulations, both topical and internal, for use by human beings. The compositions are constitute an improvement in that they replace water with Hyaluronic Acid (also known as Hyaluronan or Hyaluronate or HA) as the primary ingredient, out-performing water in hydrating the skin, minimizing TEWL, enhancing skin permeation, improving cell barrier and tissue absorption, and creating a near one hundred percent therapeutic or active formulation.

HA is a polysaccharide, more specifically, an anionic, non-sulfated glycosaminoglycan that occurs naturally in almost every tissue of the body, with the highest concentrations occurring in skin, eyes and cartilage. HA not only provides protection to the cell structure, but also act as a water reservoir for the cells and bind up to thousand times its weight in water. While the body naturally produces HA, the level of HA decreases with age. Topical application of HA can help hydrate the skin's intercellular reservoir and increase the skin's moisture level. HA can be cross linked with other therapeutic or active ingredients, enhancing skin permeation and delivery. Internal use of HA can improve the ability of the formulation to transport active ingredients through cellular barriers, across membranes, and throughout tissues.

HA may be used in a number of forms. The most common is sodium hyaluronate, which is a sodium salt hyaluronic acid. This sodium hyaluronate may be used in liquid form in which case it is already in a solution, typically with a water solvent. When the liquid form of sodium hyaluronate is used, no water is added to the formulation other than that which is in the solution of sodium hyaluronate or other solutions added to the formulation. In another for, the sodium hyaluronate way be used in a powder form, in which case it is reconstituted by adding water or other water-soluble material or botanical extract. When reconstituted in this form, no water is added to the formulation other than what may be found in the reconstituted solution or other solution added to the formulation.

The topical formulation may be further enhanced by using a combination of two or more different fractions of HA, as well as the addition of glucosamine and/or ceramides to further enhance skin permeation and promote wound healing.

In another embodiment of the invention, non-soap based emulsifiers are used which aid in penetrating the skin's protective barrier to safely and effectively deliver active or therapeutic ingredients. By using primarily ingredients that are naturally found in the body, the invention can mimic the skin structure, increase skin permeation, improve skin hydration, does not disrupt skin pH, in turn, promoting skin homeostasis and provides a superior topical composition.

Other embodiments may include a variety of therapeutic or active cosmetic, dermatological, Over-The-Counter (OTC) drug monographed, and/or pharmaceutical ingredients.

Accordingly, the present invention is directed to a Hyaluronic Acid (HA) based formulation, either topical or internal, where water has been replaced with HA as the primary ingredient of the formulation typically 50% or more of the overall formulation. The formulation may consist of one molecular weight of HA or a blend of more than one molecular weight HA. A topical composition may further comprise ceramides and glucosamine. Moreover, the topical composition may further comprise therapeutic or active dermatologic ingredients including, but are not limited to, anti-acne agents, anti-hair loss agents, anti-inflammatories, anti-oxidants, anti-wrinkle agents, botanical and organic extracts, colorants, cooling agents, emollients, firming agents, hair growth promoters, heating agents, hydroxyl acids, humectants, hydrating agents, lipo-regulators, melanin regulators, minerals, nutrients, peptides, self-tanning agents, sensory agents, sunscreen agents, tanning agents, tan enhancers, UV protectors, vitamins and other dermatologically or cosmetically effective ingredients. The topical composition may further comprise Over-The-Counter (OTC) monographed ingredients, including, but are not limited to, anti-acne agents, skin protectants, sun protectants, SPFs and/or topical analgesics.

In the present invention, several compositions are disclosed below utilizing one or more different molecular weights of HA as the primary ingredient, in place of water, along with other therapeutic or active ingredients for specific targeted benefits.

The different molecular weights of HA provide different levels of penetrating in to and hydrating of the different skin layers:

Low Molecular Weight HA (LMW-HA), having a molecular weight of under 1,500,000 Daltons, more preferably between 750,000-1,250,000 Daltons, creating a clear gel matrix that hydrates and provides lubricity on the surface of the skin.

Super-Low Molecular Weight HA (SLMW-HA), having a molecular weight of under 100,000 Daltons, more preferably between 50,000-75,000 Daltons, merging in to the skin layers and increasing moisture retention capacity over an extended period of time.

Ultra-Low Molecular Weight HA (ULMW-HA), having a molecular weight of under 10,000 Daltons, more preferably between 3,000-8,000 Daltons, unlike the other molecular weight HA, ULMW does not increase viscosity, but does penetrate in to the epidermis layer to provide benefits from the inside out.

The different molecular weight HA is cross-linked with glucosamine, more preferably N-acetylglucosamine (NAG), which triggers the natural production of the HA in the body (Kubomura and Matahira, 2006), and ceramides, which comprises 50% of the lipid domain of the SC. By combining the key ingredients naturally found in the skin, the permeation of the topical composition can be improved, as well enhanced skin repair and without disrupting skin pH, in turn promoting skin homeostasis.

Examples of therapeutic or active dermatologic ingredients suitable for use in the present invention include, but are not limited to, anti-acne agents, anti-hair loss agents, anti-inflammatories, anti-oxidants, anti-wrinkle agents, botanical and organic extracts, colorants, cooling agents, emollients, firming agents, hair growth promoters, heating agents, hydroxyl acids, humectants, hydrating agents, lipo-regulators, melanin regulators, minerals, nutrients, peptides, self-tanning agents, sensory agents, sunscreen agents, tanning agents, tan enhancers, UV protectors, vitamins and other dermatologically or cosmetically effective ingredients that are stable in the topical composition.

### EXAMPLE 1

### Anti-Wrinkle Serum

The topical composition is comprised of 25% to 90% by weight of HA, more preferably about 85%, with the 85% of the HA comprised of 30% to 90% of LMW-HA, more preferably 75% to 80%; 5% to 20% of SLMW-HA, more preferably 18%; and 1% to 5% of ULMW-HA, more preferably 1% to 2%; therapeutic anti-wrinkle peptides, proteins and natural extracts, more preferably 12-14%; a pH adjuster to reduce the pH to closer match the extracellular layer, more preferably ascorbic acid or lactic acid or another to lower the pH to a range of 4.5 to 5.0; and a method of preserving the composition, more preferably 0.5% to 1.5% of a combination of Sodium Benzoate and Potassium Sorbate or Benzyl Alcohol and Dehydroacetic Acid.

### EXAMPLE 2

### Face or Body Moisturizer

Another embodiment is comprised of 20% to 90% by weight of HA, more preferably about 40-60%, with the 40-60% of the HA comprised of 30% to 90% of LMW-HA, more preferably 65% to 80%; 10% to 20% of SLMW-HA; and 1% to 5% of ULMW-HA, more preferably 1% to 2%; a cleansing agent and foaming agent; a rheology modifier, such as a carbomer, cellulose, xanthan gum or others; 1% to 5% of an emulsifier, preferably a non-soap based emulsifier, more preferably, lecithin or a form of lecithin; 2% to 8% of a combination of emollients, including, but not limited to, caprylic/capric triglyceride, squalane, natural oils, butters; 2% to 10% of skin enhancing therapeutic cosmetic ingredients; an aromatic blend of essential oils and extracts; a pH adjuster to reduce the pH to closer match the extracellular layer, more preferably ascorbic acid or lactic acid or another to lower the pH to a range of 4.5 to 5.5; and a method of preserving the composition, more preferably 0.5% to 1.5% of a combination of Sodium Benzoate and Potassium Sorbate or Benzyl Alcohol and Dehydroacetic Acid.

### EXAMPLE 3

### Face Cleansing Gel

Another embodiment is comprised of 25% to 90% by weight of HA, more preferably about 75%, with the 75% of the HA comprised of 30% to 90% of LMW-HA, more preferably 85% to 95%; 5% to 20% of SLMW-HA, more preferably 5%; and 1% to 5% of ULMW-HA, more preferably 1% to 2%; a cleansing agent and foaming agent; a rheology modifier, such as a carbomer, cellulose, xanthan gum or others; a pH adjuster to reduce the pH to closer match the extracellular layer, more preferably ascorbic acid or lactic acid or another to lower the pH to a range of 4.5 to 5.0; and a method of preserving the composition, more preferably 0.5% to 1.5% of a combination of Sodium Benzoate and Potassium Sorbate or Benzyl Alcohol and Dehydroacetic Acid. Another embodiment of the topical composition maybe further enhanced with the incorporation of 0.1% to 25% of a physical exfoliator, including, but not limited to crushed rice, almonds, walnuts, seeds, wax beads or jojoba beads.

### EXAMPLE 4

### Skin Protectant Spray or Gel

Whether making cosmetic claims or OTC claims, another embodiment of the invention uses an OTC monographed skin protectants, more preferably allantoin. The topical composition is comprised of 25% to 90% by weight of HA, more preferably about 60 to 80%, with the HA component comprised of 10% to 90% of LMW-HA, more preferably 20% to 40%; 10% to 70% of SLMW-HA, more preferably 30% to 60%; and 1% to 35% of ULMW-HA, more preferably 10% to 20%; 0.5% to 2% of allantoin, more preferably 0.5% to 1%; a pH adjuster to reduce the pH to closer match the extracellular layer, more preferably ascorbic acid or lactic acid or another to lower the pH to a range of 4.5 to 5.0; and a method of preserving the composition, more preferably 0.5% to 1.5% of a combination of Sodium Benzoate and Potassium Sorbate or Benzyl Alcohol and Dehydroacetic Acid. An enhancement of the invention produces a gel format by adjusting the ratio of the different molecular weights of the HA and the addition of 0.01% to 1% of a rheology modifier.

### EXAMPLE 5

### Pain Relief Spray or Gel

Whether making cosmetic claims or OTC claims, another embodiment of the invention uses an OTC monographed topical analgesics, including, but not limited to, menthol, camphor, capsaicin, or others. The topical composition is comprised of 25% to 90% by weight of HA, more preferably about 60 to 80%, with the HA component comprised of 10% to 90% of LMW-HA, more preferably 20% to 40%; 10% to 70% of SLMW-HA, more preferably 30% to 60%; and 1% to 35% of ULMW-HA, more preferably 10% to 20%; OTC monographed topical analgesic; a pH adjuster to reduce the pH to closer match the extracellular layer, more preferably ascorbic acid or lactic acid or another to lower the pH to a range of 4.5 to 5.0; and a method of preserving the composition, more preferably 0.5% to 1.5% of a combination of Sodium Benzoate and Potassium Sorbate or Benzyl Alcohol and Dehydroacetic Acid. An enhancement of the invention produces a gel format by adjusting the ratio of the different molecular weights of the HA and the addition of 0.01% to 1% of a rheology modifier.

This example of embodiments can also be used as a pre- and post-workout formulation, or other products where pain relief or soothing properties are beneficial.

### EXAMPLE 6

### Hydrating, Soothing, Relieving Gel or Spray

Whether making cosmetic claims or OTC claims, another embodiment of the invention maybe further enhanced with the use OTC monographed skin protectants, more preferably allantoin; and/or an OTC monographed topical analgesic, more preferably a quaternary amine, strong ammonia solution.

The topical composition is comprised of 25% to 90% by weight of HA, more preferably about 60 to 80%, with the HA component comprised of 10% to 90% of LMW-HA, more preferably 20% to 40%; 1 0% to 70% of SLMW-HA, more preferably 30% to 60%; and 1% to 35% of ULMW-HA, more preferably 10% to 20%; 0.5% to 2% of allantoin, more preferably 0.5% to 1%; 0.5% to 5% of a OTC monographed topical analgesic, more preferably, 1.5% to 2.5% strong ammonia solution; a pH adjuster to reduce the pH to closer match the extracellular layer, more preferably ascorbic acid or lactic acid or another to lower the pH to a range of 4.5 to 5.0; and a method of preserving the composition, more preferably 0.5% to 1.5% of a combination of Sodium Benzoate and Potassium Sorbate or Benzyl Alcohol and Dehydroacetic Acid. An enhancement of the invention produces a gel format by adjusting the ratio of the different molecular weights of the HA and the addition of 0.01% to 1% of a rheology modifier.

This example of embodiments can be used for chemotherapy and radiation treatment patients to address the side effects from treatment, including, but not limited to, dry skin, chapped skin, radiation burns, wounds and discomfort, which can also help keep patients on treatment schedule. Additionally, this example of embodiments provides solutions for problems associated with diabetes, pain relief, sports injuries, back pain, pre- and post-workout, wound healing and post-surgery.

### EXAMPLE 7

### Internal Formulation

Although various combinations of ingredients may be used for internal formulations, the following is a particularly preferred formulation. HA is provided in amounts between 80% and 100% of the formulation using different molecular weights as described above. Preservatives and pH adjusters as described above may also be used. For the internal formulation, the preservatives are preferably used in 0.1% by weight of the formulation. The pH adjuster is preferably used so as to bring the pH of the formulation to below 4.0.

Botanical extracts such as aloe vera and/or green tea may be provided in amounts ranging from 0.01% to 5.0% by weight. Glycerin may be provided in a range of 2% to 10% by weight, preferably at about 5%. Phytoceramides may be used from 0.01% to 5% by weight, preferably less than 2%. Sweetener such as Stevia may be used in quantities less than 1% by weight, more specifically 0.25% to 0.5%. Other embodiments may include melatonin from 0.1% to 1.0% by weight, preferably 0.2% to 0.4%.

Although several embodiments have been described in detail for purposes of illustration, various modifications may be made without departing from the scope and spirit of the invention. Accordingly, the invention is not to be limited, except as by the appended claims.

What is claimed is:

1. A biologic formulation, comprising:
20% to 95% by weight of hyaluronic acid, said hyaluronic acid comprising the most common ingredient in the formulation;
0. 1% to 1.5% by weight of a preservative; and
a pH adjuster configured to provide a pH of less than 5.0 in the formulation.

2. The biologic formulation of claim 1, wherein the hyaluronic acid comprises a combination of a first molecular weight hyaluronic acid, a second molecular weight hyaluronic acid, and a third molecular weight hyaluronic acid.

3. The biologic formulation of claim 2, wherein the first molecular weight hyaluronic acid comprises hyaluronic acid having a molecular weight less than 1,500,000 Daltons, wherein the second molecular weight hyaluronic acid comprises hyaluronic acid having a molecular weight less than 100,000 Daltons, and wherein the third molecular weight hyaluronic acid comprises hyaluronic acid having a molecular weight less than 10,000 Daltons.

4. The biologic formulation of claim 3, wherein the first molecular weight hyaluronic acid comprises hyaluronic acid having a molecular weight between 750,000 Daltons and 1,250,000 Daltons, wherein the second molecular weight hyaluronic acid comprises hyaluronic acid having a molecular weight between 50,000 Daltons and 75,000 Daltons, and wherein the third molecular weight hyaluronic acid comprises hyaluronic acid having a molecular weight between 3,000 Daltons and 8,000 Daltons.

5. The biologic formulation of claim 2, wherein the hyaluronic acid comprises 30% to 90% of the first molecular weight hyaluronic acid, 5% to 20% of the second molecular weight hyaluronic acid, and 1% to 5% of the third molecular weight hyaluronic acid.

6. The biologic formulation of claim 2, wherein the hyaluronic acid comprises 10% to 90% of the first molecular weight hyaluronic acid, 10% to 70% of the second molecular weight hyaluronic acid, and 1% to 35% of the third molecular weight hyaluronic acid.

7. The biologic formulation of claim 2, wherein the first molecular weight, second molecular weight, and third molecular weight hyaluronic acid is cross-linked with glucosamine and ceramides.

8. The biologic formulation of claim 1, further comprising a rheology modifier selected from the group consisting of carbomer, cellulose, and xantham gum.

9. The biologic formulation of claim 8, wherein the rheology modifier comprises 0.01% to 1% by weight of the formulation.

10. The biologic formulation of claim 1, further comprising:
1% to 5% by weight of a non-soap based emulsifier; and
2% to 8% by weight of emollients.

11. The biologic formulation of claim 10, wherein the non-soap based emulsifier comprises lecithin, and the emollients comprise a combination of caprylic/capric triglyceride, squalane, natural oils, and butters.

12. The biologic formulation of claim 1, further comprising 0.1% to 25% by weight of a physical exfoliator, wherein the physical exfoliator comprises crushed rice, almonds, walnuts, seeds, wax beads, or jojoba beads.

13. The biologic formulation of claim 1, further comprising 0.5% to 2% by weight of allantoin.

14. The biologic formulation of claim 1, further comprising 0.5% to 5% by weight of a monographed topical analgesic.

15. The biologic formulation of claim 14, wherein the topical analgesic comprises a strong ammonia solution.

16. The biologic formulation of claim 1, wherein the preservative comprises a combination of sodium benzoate and potassium sorbate, or benzyl alcohol and dehydroacetic acid.

17. The biologic formulation of claim 1, wherein the pH adjuster comprises ascorbic acid or lactic acid.

18. The biologic formulation of claim 1, wherein the biologic formulation is an internal formulation comprising 60% to 95% by weight of hyaluronic acid, 0.1% of the preservative, and pH adjuster configured to lower the pH to less than 4.0.

19. The biologic formulation of claim 18, further comprising 0.01% to 5% of botanical extracts, 2% to 10% of glycerin, and 0.01% to 5% phytoceramides.

20. The biologic formulation of claim 18, further comprising 0.1% to 1.0% of melatonin.
